(19) United States
(12) Patent Application Publication
(10) Pub. No.: US 2008/0200481 A1
(43) Pub. Date: Aug. 21, 2008

(54) Method of treatment of myocardial infarction

(75) Inventors: David A. Cheresh, Encinitas, CA (US); Robert Paul, Munich (DE); Brian Eliceiri, Carlsbad, CA (US)

Correspondence Address: Olson & Cepuritis, LTD., 20 NORTH WACKER DRIVE, 36TH FLOOR, CHICAGO, IL, 60606, US

(73) Assignee: The Scripps Research Institute

(21) Appl. No.: 12/148,001

(22) Filed: Apr. 16, 2008

Related U.S. Application Data
(63) Continuation of application No. 10/535,325, filed on May 18, 2005.
Application No. 10/535,325 is a continuation-in-part of application No. 10/298,377, filed on Nov. 18, 2002.
Application No. 10/298,377 is a continuation-in-part of application No. 09/538,248, filed on Mar. 29, 2000, now abandoned.
Application No. 09/538,248 is a continuation-in-part of application No. 09/470,881, filed on Dec. 22, 1999, now Pat. No. 6,685,938.
Application No. 09/470,881 is a continuation-in-part of application No. PCT/US99/11780, filed on May 28, 1999.
(60) Provisional application No. 60/087,220, filed on May 29, 1998.

(51) Int. Cl. A61K 31/519; A61P 9/10

(52) U.S. Cl. 514/262.1; 514/264.11

(57) ABSTRACT

Myocardial infarction in a mammal is treated by administering to the mammal a therapeutically effective amount of a chemical Src family tyrosine kinase protein inhibitor and the use of such inhibitor compounds for the preparation of a medicament for treating myocardial infarction. Myocardial infarction can be prevented by administering to the mammal a prophylactic amount of the inhibitor. The inhibitor preferably is an inhibitor of Src protein selected from the group consisting of a pyrazolopyrimidine class Src family tyrosine kinase inhibitor, a macrocyclic dienone class Src family tyrosine kinase inhibitor, a pyrido[2,3-d]pyrimidine class Src family tyrosine kinase inhibitor, a 4-anilino-3-quinolinecarbonitrile class Src family tyrosine kinase inhibitor, and a mixture thereof. The Src family tyrosine kinase inhibitors can be used to prepare medicaments for the treatment of myocardial infarction. Also disclosed are articles of manufacture containing a chemical Src family tyrosine kinase inhibitor.

### CROSS-REFERENCE TO RELATED APPLICATIONS

This application is a continuation of U.S. patent application Ser. No. 10/535,325, filed on May 18, 2005, which is a continuation-in-part of U.S. patent application Ser. No. 10/298,377, filed on Nov. 18, 2002, which is a continuation-in-part of U.S. patent application Ser. No. 09/538,248, filed on Mar. 29, 2000, which is a continuation-in-part of U.S. patent application Ser. No. 09/470,881, filed on Dec. 22, 1999, which in turn is a continuation-in-part of International Patent Application Number PCT/US99/11780, designating the United States of America and filed May 29, 1998, which claims the benefit of U.S. Provisional Application for Patent Ser. No. 60/087,220, filed May 29, 1998. The complete disclosures of these applications are incorporated herein by reference.

### STATEMENT OF GOVERNMENT RIGHTS

This invention was made with governmental support under contract numbers CA 50286, CA 45726, CA 75924, CA 78045, HL 54444, and HL 09435 by the National Institutes of Health. The government has certain rights in this invention.

### TECHNICAL FIELD

The present invention relates generally to the field of medicine, and relates specifically to methods and compositions for treating myocardial infarction in mammals.

### BACKGROUND

Vascular permeability due to injury, disease, or other trauma to the blood vessels is a major cause of vascular leakage and edema associated with tissue damage. For example, cerebrovascular disease associated with cerebrovascular accident (CVA) or other vascular injury in the brain or spinal tissues are the most common cause of neurologic disorder, and a major source of disability. Typically, damage to the brain or spinal tissue in the region of a CVA involves vascular leakage and/or edema. Typically, CVA can include injury caused by brain ischemia, interruption of normal blood flow to the brain; cerebral insufficiency due to transient disturbances in blood flow; infarction, due to embolism or thrombosis of the intra- or extracranial arteries; hemorrhage; and arteriovenous malformations. Ischemic stroke and cerebral hemorrhage can develop abruptly, and the impact of the incident generally reflects the area of the brain damaged. (See The Merck Manual, 16^{th} ed. Chp. 123, 1992).

Other than CVA, central nervous system (CNS) infections or disease can also affect the blood vessels of the brain and spinal column, and can involve inflammation and edema, as in for example bacterial meningitis, viral encephalitis, and brain abscess formation. (See The Merck Manual, 16^{th} ed. Chp. 125, 1992). Systemic disease conditions can also weaken blood vessels and lead to vessel leakage and edema, such as diabetes, kidney disease, atherosclerosis, myocardial infarction, and the like. Thus, vascular leakage and edema are critical pathologies, distinct from and independent of cancer, which are in need of effective specific therapeutic intervention in association with a variety of injury, trauma or disease conditions.

Myocardial infarction is the death of heart tissue due to an occluded blood supply to the heart muscles. Myocardial infarction is one of the most common diagnoses in hospitalized patients in western countries. It has been reported that about 1.1 million people in the United States are diagnosed with acute myocardial infarction per year. Mortality from myocardial infraction can be over 53%, and as many as 66% of the surviving patients fail to achieve full recovery. A reduction of just one percent in mortality could save as many as 3400 lives per year. Myocardial infarction and attendant edema generally occur when a coronary artery is occluded, cutting off the supply of oxygen to the heart tissue supplied by the blocked artery. When the blood supply is blocked, the tissue normally supplied with blood by the blocked artery becomes ischemic. Eventually the oxygen-deprived heart tissue begins to die off (necrosis). Honkanen et al., in U.S. Pat. No. 5,914,242, describe a method for diminishing myocardial infarction comprising administering certain serine/threonine phosphatase enzyme inhibitors and related polypeptides to a patient after the onset of cardiac ischemia. Such enzymes and polypeptides are expensive and complicated to manufacture and purify for pharmaceutical use.

We have discovered that inhibition of Src family tyrosine kinase activity provides a useful method for treatment of myocardial infarction, by reducing edema and the resulting necrosis of coronary tissue that normally results from occlusion of coronary vasculature, thereby alleviating the tissue damaging effects of myocardial infarction.

### SUMMARY OF THE INVENTION

The present invention is directed to a method of treatment of myocardial infarction (MI) by inhibition of Src family tyrosine kinase activity. The method involves treating the coronary tissue of a mammal suffering from coronary vascular occlusion with an effective amount of an inhibitor of a Src family tyrosine kinase. The mammal can be a human patient or a non-human mammal. The coronary tissue to be treated can be any be any portion of the heart that is suffering from ischemia (i.e. loss of blood flow) due to coronary vascular occlusion. Therapeutic treatment is accomplished by contacting the target coronary tissue with an effective amount of the desired pharmaceutical composition comprising a chemical (i.e., non-peptidic) Src family tyrosine kinase inhibitor. It is useful to treat diseased coronary tissue in a region near where deleterious vascular occlusion is occurring or has occurred. The method provides a reduction in tissue necrosis (infarction) normally resulting from a coronary vascular occlusion.

A further aspect of the present invention is an article of manufacture which comprises packaging material and a pharmaceutical composition contained within the packaging material, wherein the pharmaceutical composition is capable of reducing necrosis in a coronary tissue suffering from a loss of blood flow due to coronary vascular occlusion. The packaging material comprises a label that indicates that the pharmaceutical composition can be used for treating myocardial infarction, and that the pharmaceutical composition comprises a therapeutically effective amount of a Src family tyrosine kinase inhibitor in a pharmaceutically acceptable carrier.

Suitable Src family tyrosine kinase inhibitors for purposes of the present invention include the pyrazolopyrimidine class of Src family tyrosine kinase inhibitors, such as 4-amino-5-(4-methylphenyl)-7-(t-butyl)pyrazolo[3,4-d-]pyrimidine (AGL 1872), 4-amino-5-(4-chlorophenyl)-7-(t-butyl)pyrazolo[3,4-d-]pyrimidine (AGL 1879), and the like; the macrocyclic dienone class of Src family tyrosine kinase inhibitors, such as Radicicol R2146, Geldanamycin, Herbimycin A, and the like; the pyrido[2,3-d]pyrimidine class of Src family tyrosine kinase inhibitors, such as PD173955, and the like; the 4-anilino-3-quinolinecarbonitrile class of Src family tyrosine kinase inhibitors, such as SKI-606, and the like; and mixtures thereof.

The methods of the present invention are useful for treating myocardial infarction. In particular, the methods of the present invention are useful for ameliorating necrosis of heart tissue due to coronary vascular blockage due to heart disease, injury, or trauma.

### BRIEF DESCRIPTION OF THE DRAWINGS

In the drawings forming a portion of this disclosure:

FIG. 1 is a cDNA sequence (SEQ ID NO: 1) of human c-Src which was first described by Braeuninger et al., Proc. Natl. Acad. Sci., USA, 88:10411-10415 (1991). The sequence is accessible through GenBank Accession Number X59932 X71157. The sequence contains 2187 nucleotides with the protein coding portion beginning and ending at the respective nucleotide positions 134 and 1486.

FIG. 2 is the encoded amino acid residue sequence of human c-Src of the coding sequence shown in FIG. 1. (SEQ ID NO: 2).

FIG. 3 depicts the nucleic acid sequence (SEQ ID NO: 3) of a cDNA encoding for human c-Yes protein. The sequence is accessible through GenBank Accession Number M15990. The sequence contains 4517 nucleotides with the protein coding portion beginning and ending at the respective nucleotide positions 208 and 1839, and translating into to the amino acid sequence depicted in FIG. 4.

FIG. 4 depicts the amino acid sequence of c-Yes (SEQ ID NO: 4).

FIG. 5 illustrates results from a modified Miles assay for VP of VEGF in the skin of mice deficient in Src, Fyn and Yes. FIG. 5A are photographs of treated ears. FIG. 5B are graphs of experimental results for stimulation of the various deficient mice. FIG. 5C plots the amount of Evan's blue dye eluted by the treated tissues.

FIG. 6 is a graph depicting the relative size of cerebral infarct in Src +/−, Src −/−, wild type (WET), and AGL1872 (i.e., 4-amino-5-(4-methylphenyl)-7-(t-butyl)pyrazolo[3,4-d-]pyrimidine) treated wild type mice. The dosage was 1.5 mg/kg body weight.

FIG. 7 depicts sequential MRI scans of control and AGL1872 treated mouse brains showing less brain infarction in AGL1872 treated animal (right) than in the control animal (left).

FIG. 8 depicts the structures of preferred pyrazolopyrimidine class Src family tyrosine kinase inhibitors of the invention.

FIG. 9 depicts the structures of preferred macrocyclic dienone Src family tyrosine kinase inhibitors of the invention.

FIG. 10 depicts the structure of a preferred pyrido[2,3-d]pyrimidine class Src family tyrosine kinase inhibitors of the invention.

FIG. 11 depicts photomicrographic images of vital stained rat heart tissue that has been traumatized to induce myocardial infarction; the image on the right is the control, showing a significant level of necrosis; the image on the left is tissue treated with a chemical Src family tyrosine kinase inhibitor (AGL1872), showing a dramatically reduced level of necrosis.

FIG. 12 depicts a bar graph of the size of myocardial infarct as a function of inhibitor (AGL1872) concentration.

FIG. 13 depicts a bar graph of the size of myocardial infarct as a function of time after treatment with inhibitor (AGL1872).

FIG. 14 depicts a bar graph of myocardial water content as a function of inhibitor (AGL1872) concentration.

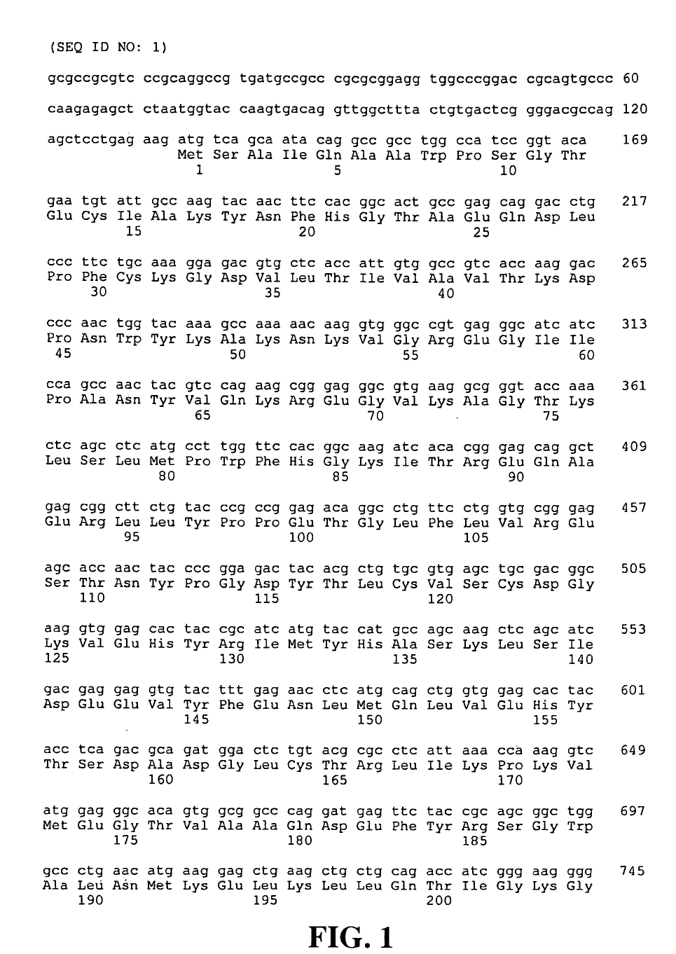

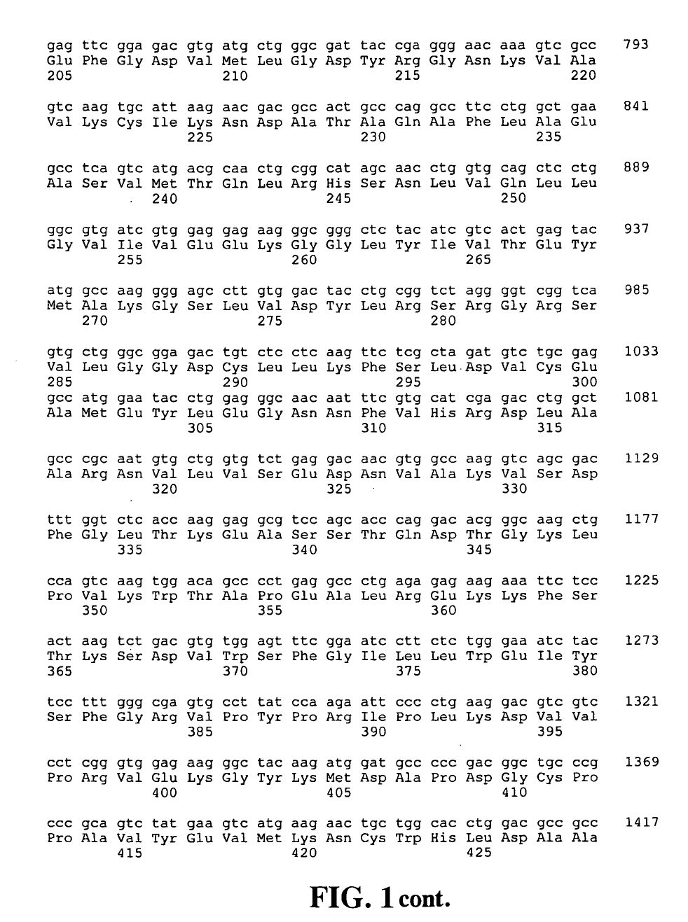

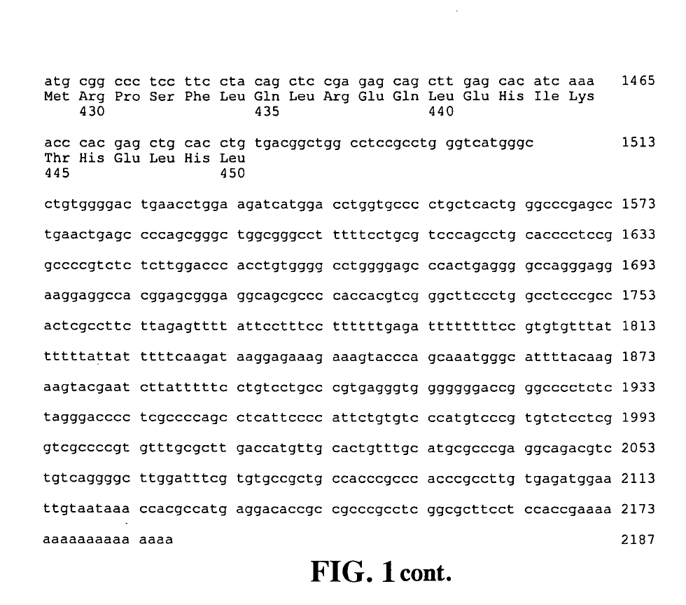

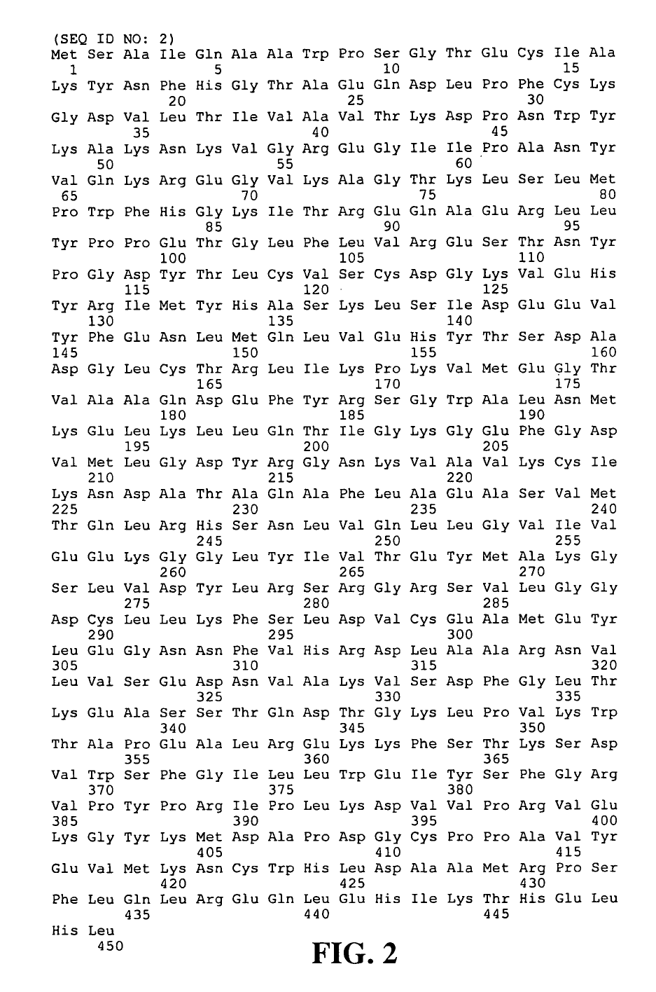

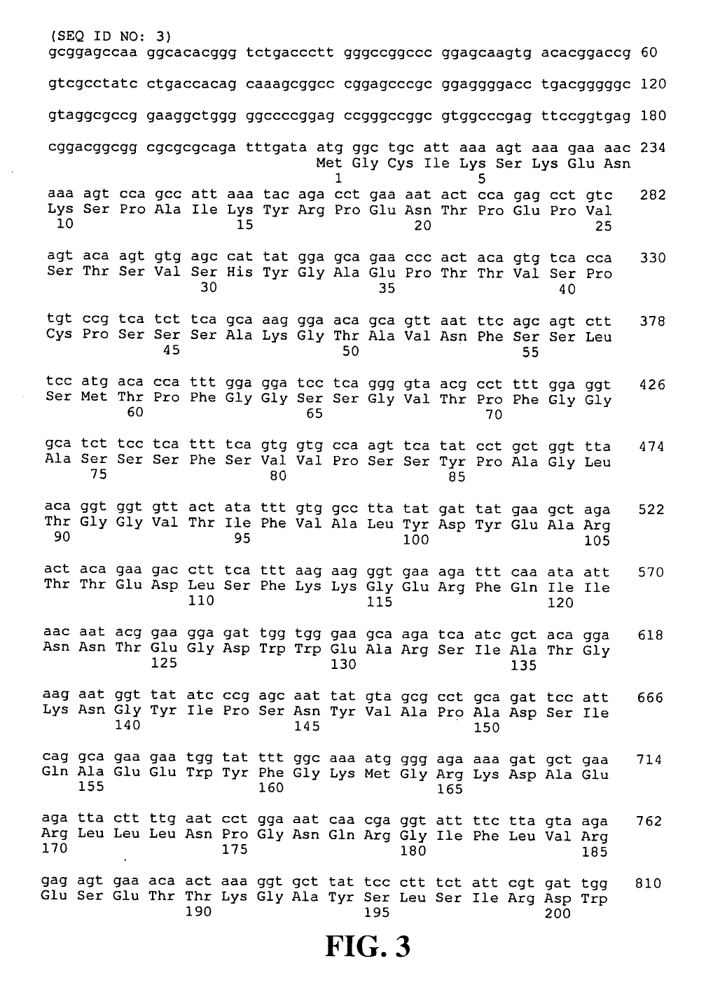

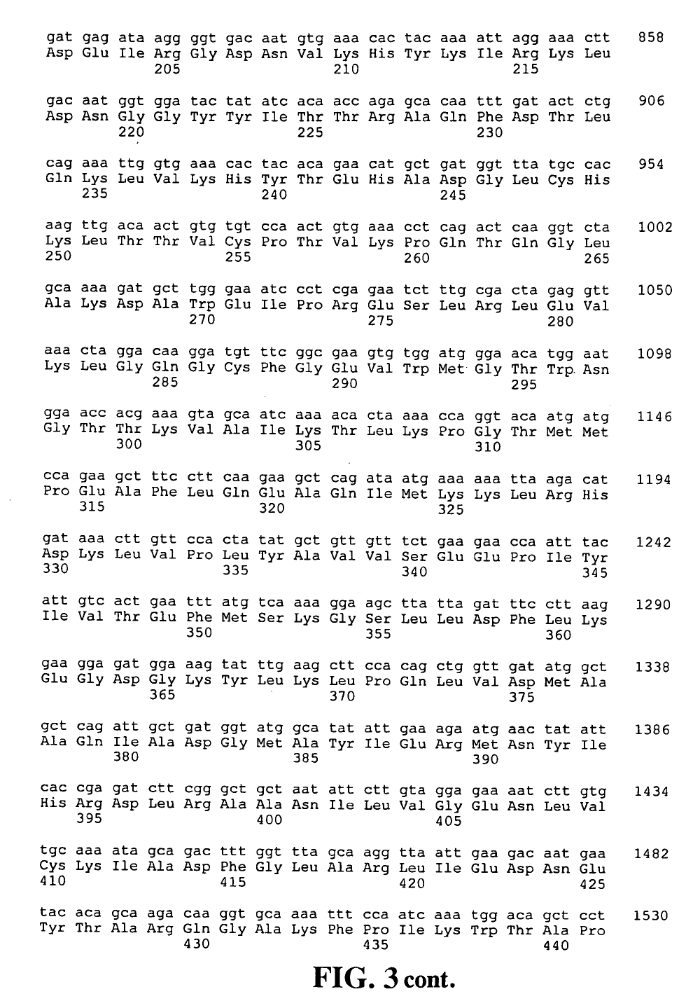

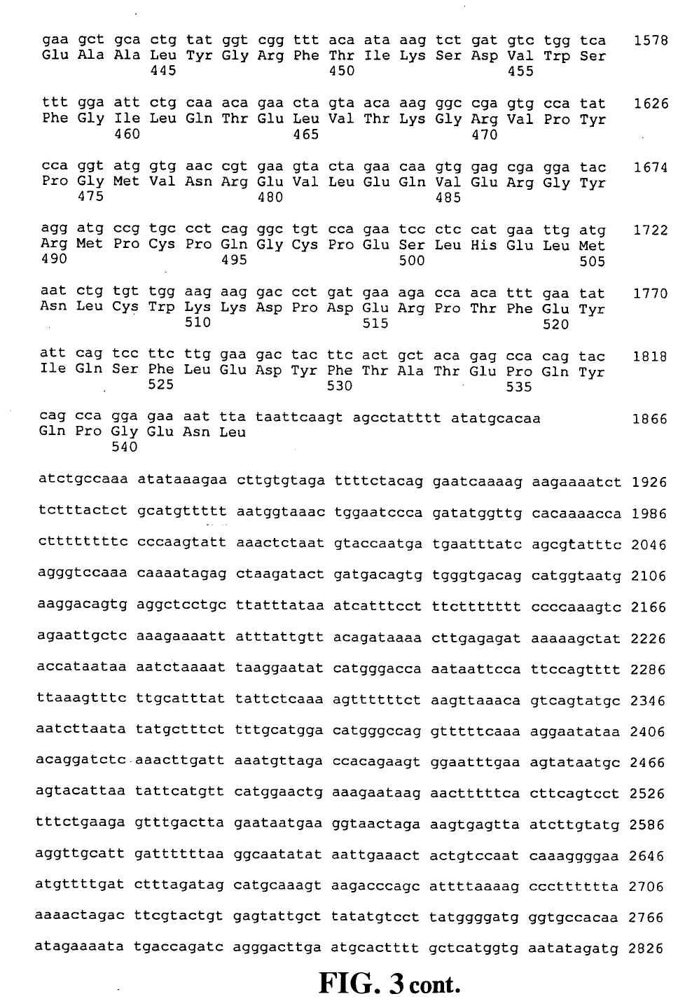

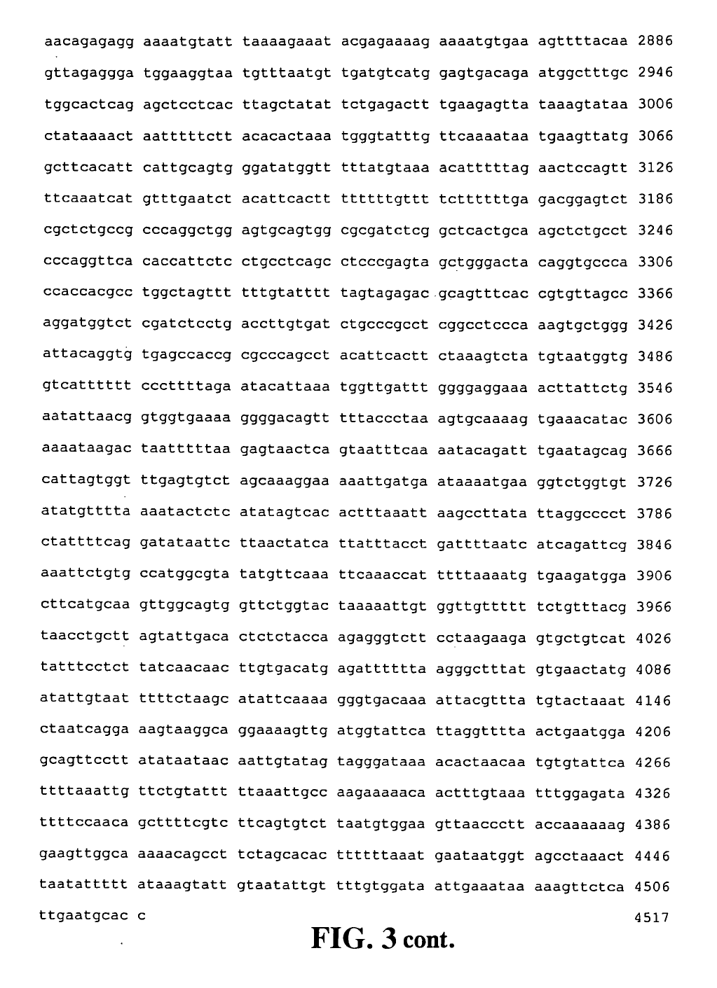

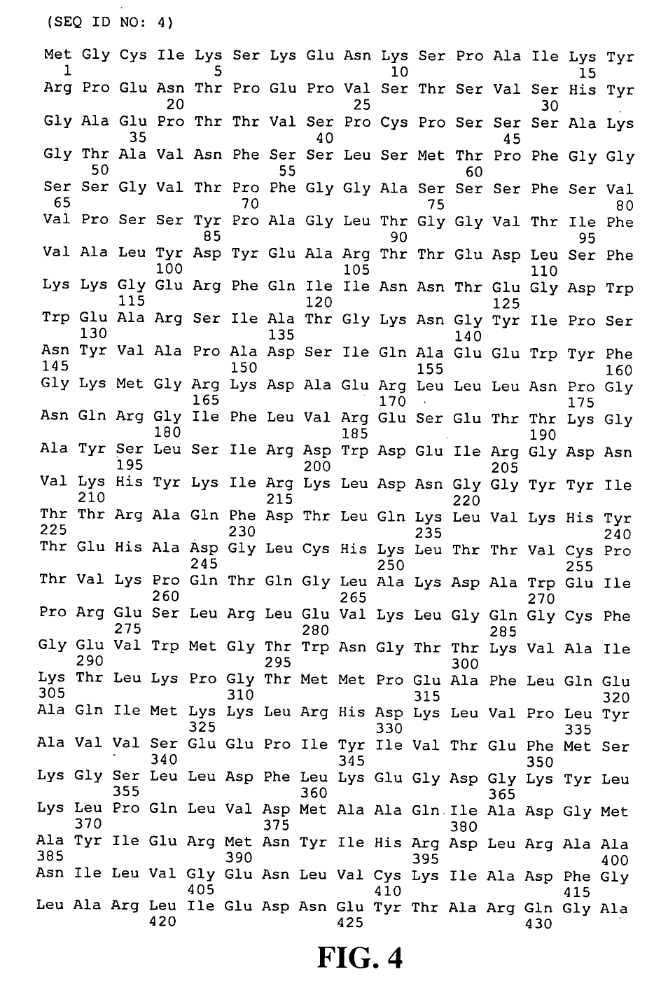

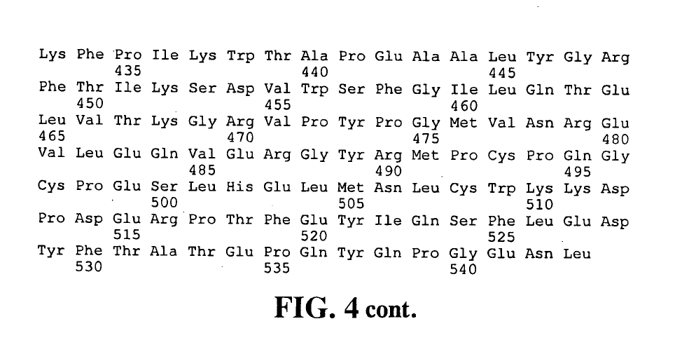

### DETAILED DESCRIPTION OF THE INVENTION

### A. Definitions

The term “amino acid residue”, as used herein, refers to an amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxyl group present at the carboxyl terminus of a polypeptide in keeping with standard polypeptide nomenclature (described in J. Biol. Chem., 243:3552-59 (1969) and adopted at 37 CFR §1.822(b)(2)).

It should be noted that all amino acid residue sequences are represented herein by formulae whose left and right orientation is in the conventional direction of amino-terminus (N-terminus) to carboxyl-terminus (C-terminus). Furthermore, it should be noted that a dash at the beginning or end of an amino acid residue sequence indicates a peptide bond to a further sequence of one or more amino acid residues.

The term “polypeptide”, as used herein, refers to a linear series of amino acid residues connected to one another by peptide bonds between the alpha-amino group and carboxyl group of contiguous amino acid residues.

The term “peptide”, as used herein, refers to a linear series of no more than about 50 amino acid residues connected one to the other as in a polypeptide.

The term “protein”, as used herein, refers to a linear series of greater than 50 amino acid residues connected one to the other as in a polypeptide.

### B. General Considerations

The present invention relates generally to: (1) the discovery that VEGF induced vascular permeability (VP) is specifically mediated by tyrosine kinase proteins such as Src and Yes, and that VP can be modulated by inhibition of Src family tyrosine kinase activity; and (2) the discovery that in vivo administration of a Src family tyrosine kinase inhibitor decreases tissue damage due to disease- or injury-related increase in vascular permeability.

This discovery is important because of the role that vascular permeability plays in a variety of disease processes. The present invention relates to the discovery that vascular permeability can be specifically modulated, and ameliorated, by inhibition of Src family tyrosine kinase activity. In particular, the present invention is related to the discovery that the in vivo administration of a Src family tyrosine kinase inhibitor decreases tissue damage due to disease- or injury-related increase in vascular permeability that is not associated with cancer or angiogenesis.

Vascular permeability is implicated in a variety of disease processes where tissue damage is caused by the sudden increase in VP due to trauma to the blood vessel. Thus, the ability to specifically modulate VP allows for novel and effective treatments to reduce the adverse effects of stroke.

Examples of tissue associated with disease or injury induced vascular leakage and/or edema that will benefit from the specific inhibitory modulation using a Src family kinase inhibitor include rheumatoid arthritis, diabetic retinopathy, inflammatory diseases, restenosis, stroke, myocardial infarction, and the like.

It has been reported that systemic neutralization of VEGF protein using a VEGF receptor IgG fusion protein reduces infarct size following cerebral ischemia. This effect was attributed to the reduction of VEGF-mediated vascular permeability. N. van Bruggen et al., J. Clin. Inves. 104:1613-1620 (1999). However, VEGF is not the critical mediator of vascular permeability increase that Src has now been discovered to be. Moreover, Src can be activated by stimuli other than VEGF. See for example, Erpel et al., Cell Biology, 7:176-182 (1995).

The present invention relates, in particular, to the discovery that Src family tyrosine kinase inhibitors, particularly inhibitors of Src, are useful for treating myocardial infarction by ameliorating coronary tissue damage in a mammal due to coronary vascular occlusions.

### C. Src Family Tyrosine Kinase Proteins

As used herein and in the appended claims, the term “Src family tyrosine kinase protein” and grammatical variations thereof, refers in particular to a protein having an amino acid sequence homology to v-Src, N-terminal myristolation, a conserved domain structure having an N-terminal variable region, followed by a SH3 domain, a SH2 domain, a tyrosine kinase catalytic domain and a C-terminal regulatory domain. The terms “Src protein” and “Src” are used to refer collectively to the various forms of tyrosine kinase Src protein having a 60 kDa molecular weight, an N-terminal variable region including 2 PKC phosphorylation sites and one PKA phosphorylation site, a relatively higher overall amino acid sequence identity to known Src proteins than to known members of other Src-family subgroups (e,g., Yes, Fyn, Lck, and Lyn), and which are activated by phosphorylation of a tyrosine that is equivalent to tyrosine at position 416 in SEQ ID NO: 2. The terms “Yes protein” and “Yes” are used to refer collectively to the various forms of tyrosine kinase Yes protein having a 62 kDa molecular weight, an N-terminal variable region lacking any phosphorylation sites, a relatively higher overall amino acid sequence identity to known Yes proteins than to known members of other Src-family subgroups, (e.g., Src, Fyn, Lck, and Lyn), and which are activated by phosphorylation of a tyrosine that is equivalent to tyrosine at position 426 in SEQ ID NO: 4.

A preferred assay for measuring coronary ischemia involves inducing ischemia in rats by ligation of a coronary artery and assessing the size of myocardial infarction by MRI, echocardiography, and the like techniques, over time as described in detail herein below.

### D. Methods of Treating and Preventing Myocardial Infarction

The methods of the present invention comprise contacting ischemic coronary tissue with a pharmaceutical composition that includes at least one chemical Src family tyrosine kinase inhibitor.

Suitable Src family tyrosine kinase inhibitors for purposes of the present invention include chemical inhibitors of Src such as pyrazolopyrimidine class of Src family tyrosine kinase inhibitors, the macrocyclic dieneone class of Src family tyrosine kinase inhibitors, the pyrido[2,3-d]pyrimidine class of Src family tyrosine kinase inhibitors, and the 4-anilino-3-quinoline carbonitrile class of Src family tyrosine kinase inhibitors. Mixtures of inhibitors may also be utilized.

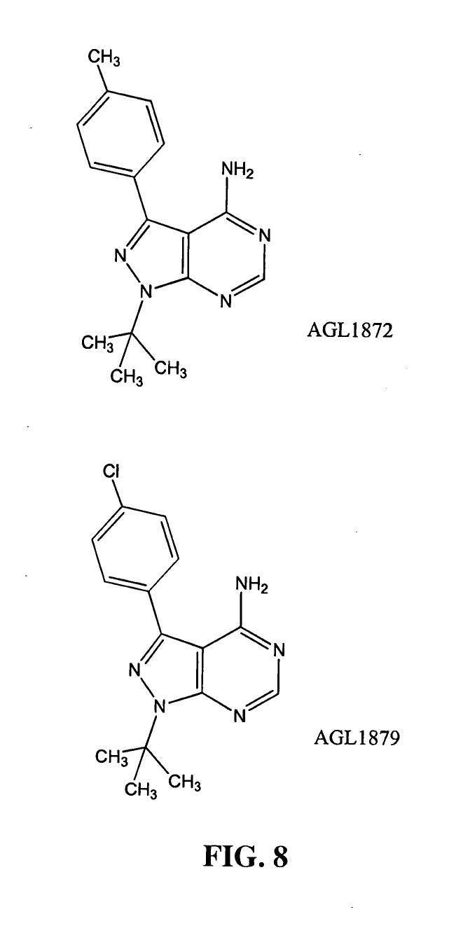

Preferred pyrazolopyrimidine class inhibitors include, 4-amino-5-(4-methylphenyl)-7-(t-butyl)pyrazolo[3,4-d-]pyrimidine (also sometimes referred to as PP1 or AGL1872), 4-amino-5-(4-chlorophenyl)-7-(t-butyl)pyrazolo[3,4-d-]pyrimidine (also sometimes referred to as PP2 or AGL1879), and the like, the detailed preparation of which are described in Waltenberger, et al. Circ. Res., 85:12-22 (1999), the relevant disclosure of which is incorporated herein by reference. The chemical structures of AGL1872 and AGL1879 are illustrated in FIG. 8. AGL1872 (PP1) is available from Biomol, by license from Pfizer, Inc. AGL1879 (PP2) is available from Calbiochem, on license from Pfizer, Inc. (see also Hanke et al., J. Biol. Chem. 271(2):695-701 (1996)).

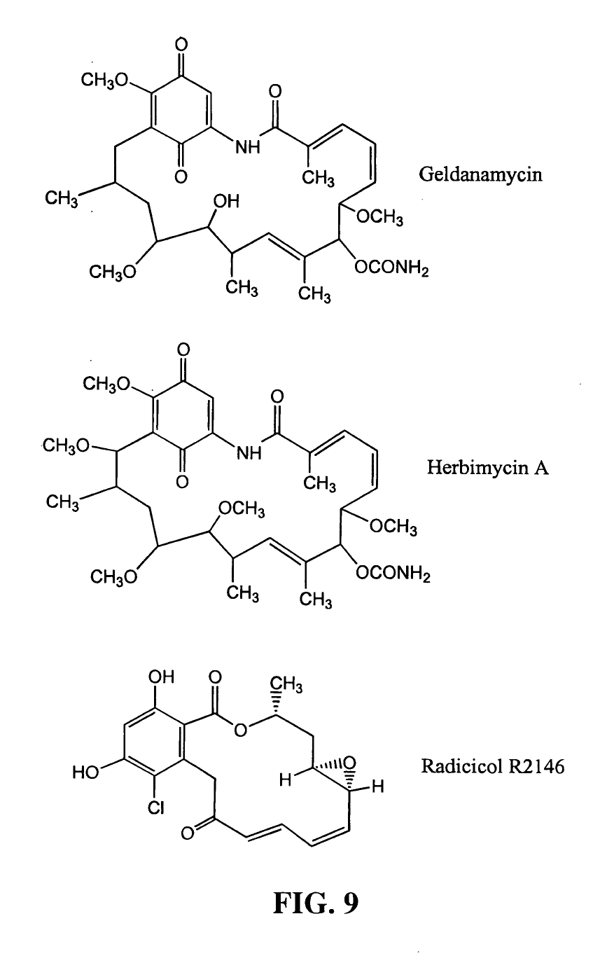

Preferred macrocyclic dienone inhibitors include, for example, Radicicol R2146, Geldanamycin, Herbimycin A, and the like. The structures of Radicicol R2146, Geldanamycin and Herbimycin A are illustrated in FIG. 9. Geldanamycin is available from Life Technologies. Herbimycin A is available from Sigma. Radicicol, which is offered commercially by different companies (e.g. Calbiochem, RBI, Sigma), is an antifungal macrocyclic lactone antibiotic that also acts as an unspecific protein tyrosine kinase inhibitor and was shown to inhibit Src kinase activity. The macrocyclic dienone inhibitors comprise a 12 to 20 carbon macrocyclic lactam or lactone ring structure containing a α,β,γ,δ-bis-unsaturated ketone (i.e. a dienone) moiety and an oxygenated aryl moiety as a portion of the macrocyclic ring.

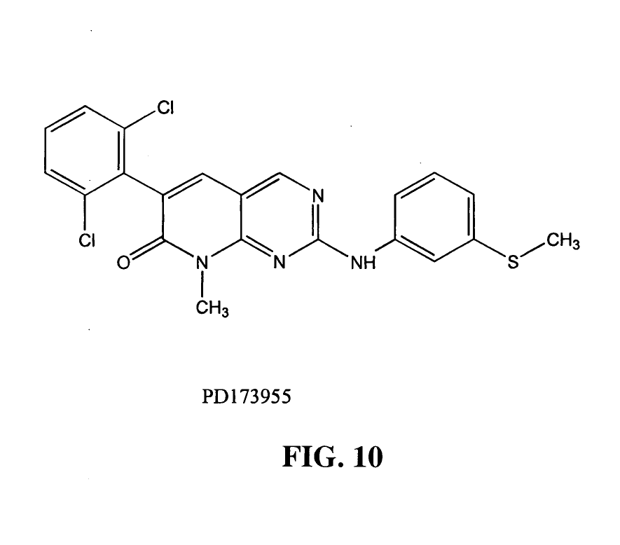

Preferred pyrido[2,3-d]pyrimidine class inhibitors include, for example PD173955 and the like. The structure of PD173955, an inhibitor developed by Parke Davis, is disclosed in Moasser, et al., Cancer Res., 59:6145-6152 (1999) the relevant disclosure of which is incorporated herein by reference. The chemical structure of PD172955 is illustrated in FIG. 10.

Preferred 4-anilino-3-quinoline carbonitrile class inhibitors, include, for example SKI-606 available from Wyeth. Examples of 4-anilino-3-quinolinecarbonitrile Src inhibitors are disclosed in U.S. Patent Publications No. 2001/0051520 and No. 2002/00260052, the relevant disclosures of which are incorporated herein by reference.

Other specific Src kinase inhibitors useful in the methods and compositions of the present invention include PD162531 (Owens et al., Mol. Biol. Cell 11:51-64 (2000)), which was developed by Parke Davis, but the structure of which is not accessible from the literature.

Preferably the chemical inhibitor is a pyrazolopyrimidine inhibitor, more preferably AGL1872 and AGL1879, most preferably the chemical inhibitor is AGL1872. Another preferred Src inhibitor is the 4-anilino-3-quinolinecarbonitrile known as SKI-606.

Additional suitable Src family tyrosine kinase inhibitors can be identified and characterized using standard assays known in the art. For example, screening of chemical compounds for potent and selective inhibitors for Src or other tyrosine kinases has been done and have resulted in the identification of chemical moieties useful in potent inhibitors of Src family tyrosine kinases.

For example, catechols have been identified as important binding elements for a number of tyrosine kinase inhibitors derived from natural products, and have been found in compounds selected by combinatorial target-guided selection for selective inhibitors of c-Src. See Maly et al. “Combinatorial target-guided ligand assembly: Identification of potent subtype-selective c-Src inhibitors” PNAS (USA)97(6):2419-2424 (2000)). Combinatorial chemistry based screening of candidate inhibitor compounds, using moieties known to be important to Src inhibition as a starting point, is a potent and effective means for isolating and characterizing other chemical inhibitors of Src family tyrosine kinases.

However, even careful selection of potential binding elements based upon the potential for mimicking a wide range of functionalities present on polypeptides and nucleic acids can be used to perform combinatorial screens for active inhibitors. For example, O-methyl oxime libraries are particularly suited for this task, given that the library is easily prepared by condensation of O-methylhydroxylamine with any of a large number of commercially available aldehydes. O-alkyl oxime formation is compatible with a wide range of functionalities which are stable at physiological pH. See Maly et al., supra.

The mammal that can be treated by a method embodying the present invention is desirably a human, although it is to be understood that the principles of the invention indicate that the present methods are effective with respect to non-human mammals as well. In this context, a mammal is understood to include any mammalian species in which treatment of vascular leakage or edema associated tissue damage is desirable, agricultural and domestic mammalian species, as well as humans.

A preferred method of treatment comprises administering to a mammal suffering from myocardial infarction a therapeutically effective amount of a physiologically tolerable composition containing a chemical Src family tyrosine kinase inhibitor, particularly a chemical (i.e., non-peptidal) inhibitor of Src.

A preferred method of preventing myocardial infarction comprises administering to a mammal at risk of myocardial infarction a prophylactic amount of a physiologically tolerable composition containing a chemical Src family tyrosine kinase inhibitor, particularly a chemical (i.e., non-peptidal) inhibitor of Src.

The dosage ranges for the administration of chemical Src family tyrosine kinase inhibitors, such as AGL1872 or SKI-606, can be in the range of about 0.1 mg/kg body weight to about 100 mg/kg body weight, or the limit of solubility of the active agent in the pharmaceutical carrier. A preferred dosage is about 1.5 mg/kg body weight. The pharmaceutical compositions embodying the present invention can also be administered orally. Illustrative dosage forms for oral administration include capsules, tablets with or without an enteric coating, and the like.

In the case of acute injury or trauma, it is best to administer treatment as soon as possible after the occurrence of the incident. However, time for effective administration of a Src family tyrosine kinase inhibitors can be within about 48 hours of the onset of injury or trauma, in the case of acute incidents. It is preferred that administration occur within about 24 hours of onset, within 6 hours being better. Most preferably the Src family tyrosine kinase inhibitor is administered to the patient within about 45 minutes of the injury. Administration after 48 hours of initial injury may be appropriate to ameliorate additional tissue damage due to further vascular leakage or edema; however, the beneficial effect on the initial tissue damage may be reduced in such cases.

Where prophylactic administration is made to prevent myocardial infarction associated with a surgical procedure, or made in view of predisposing diagnostic criteria, administration can occur prior to any actual coronary vascular occlusion, or during such occlusion causing event, for example, percutaneous cardiovascular interventions, such as coronary angioplasty. For the treatment of chronic conditions which lead to coronary vascular occlusion, administration of chemical Src family tyrosine kinase inhibitors can be made with a continuous dosing regimen.

Generally, the dosage can vary with the age, condition, sex and extent of the injury suffered by the patient, and can be determined by one of skill in the art. The dosage can also be adjusted by the individual physician in the event of any complication.

The pharmaceutical compositions of the invention preferably are administered parenterally by injection, or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, compositions of the invention can be administered intravenously, intraperitoneally, intramuscularly, subcutaneously, intracavity, transdermally, orally, and can also be delivered by peristaltic means.

Intravenous administration is effected by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

In one preferred embodiment the active agent is administered in a single dosage intravenously. Localized administration can be accomplished by direct injection or by taking advantage of anatomically isolated compartments, isolating the microcirculation of target organ systems, reperfusion in a circulating system, or catheter based temporary occlusion of target regions of vasculature associated with diseased tissues.

The pharmaceutical compositions are administered in a manner compatible with the dosage formulation, and in a therapeutically effective amount. The terms “therapeutically effective amount” and “prophylactic amount” as used herein and in the appended claims, in reference to pharmaceutical compositions, means an amount of pharmaceutical composition that will elicit the biological or medical response of a subject that is sought by a clinician (e.g., amelioration of tissue damage or prevention of myocardial infarction).

The quantity to be administered and timing depends on the subject to be treated, capacity of the subject's system to utilize the active ingredient, and degree of therapeutic effect desired. Precise amounts of active ingredient to be administered depend on the judgement of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration, e.g., oral administration. Alternatively, continuous intravenous infusion sufficient to maintain concentrations in the blood in the ranges specified for in vivo therapies are contemplated.

The methods of the invention ameliorating tissue damage due to coronary vascular occlusion associated with a various forms of coronary disease or due to injury or trauma of the heart, ameliorates symptoms of the disease and, depending upon the disease, can contribute to cure of the disease. The extent of necrosis in a tissue, and therefore the extent of inhibition achieved by the present methods, can be evaluated by a variety of methods. In particular, the methods of the present invention are eminently well suited for treatment of myocardial infarction.

Amelioration of tissue damage due to coronary vascular occlusion can occur within a short time after administration of the therapeutic composition. Most therapeutic effects can be visualized 24 hours of administration, in the case of acute injury or trauma. Effects of chronic administration will not be as readily apparent, however.

The time-limiting factors include rate of tissue absorption, cellular uptake, protein translocation or nucleic acid translation (depending on the therapeutic) and protein targeting. Thus, tissue damage modulating effects can occur in as little as an hour from time of administration of the inhibitor. The heart tissue can also be subjected to additional or prolonged exposure to Src family tyrosine kinase inhibitors utilizing the proper conditions. Thus, a variety of desired therapeutic time frames can be designed by modifying such parameters.

### E. Therapeutic Compositions

Src family tyrosine kinase inhibitors, as described herein, can be used to prepare medicaments for treatment of myocardial infarction. The inhibitors can be included in pharmaceutical compositions useful for practicing the therapeutic and prophylactic methods described herein. Pharmaceutical compositions of the present invention contain a physiologically tolerable carrier together with a chemical Src family tyrosine kinase inhibitor as described herein, dissolved or dispersed therein as an active ingredient. In a preferred embodiment, the pharmaceutical composition is not immunogenic when administered to a mammalian patient, such as a human, for therapeutic purposes.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectable, either as liquid solutions or suspensions. Solid forms suitable for solution, or suspensions, in liquid prior to use can also be prepared. The preparation can also be emulsified or presented as a liposome composition.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain amounts of auxiliary substances such as wetting or emulsifying agents, pH buffering agents and the like which enhance the effectiveness of the active ingredient.

The therapeutic composition of the present invention can include pharmaceutically acceptable salts of the active components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

Chemical therapeutic compositions of the present invention contain a physiologically tolerable carrier together with a Src family tyrosine kinase inhibitor dissolved or dispersed therein as an active ingredient.

Suitable Src family tyrosine kinase inhibitors inhibit the biological tyrosine kinase activity of Src family tyrosine kinases. A more suitable Src family tyrosine kinase has primary specificity for inhibiting the activity of the Src protein, and secondarily inhibits the most closely related Src family tyrosine kinases.

### F. Articles of Manufacture

The invention also contemplates an article of manufacture which is a labeled container for providing a therapeutically effective amount of a Src family tyrosine kinase inhibitor. The inhibitor can be a single packaged chemical Src family tyrosine kinase inhibitor, or combinations of more than one inhibitor. An article of manufacture comprises packaging material and a pharmaceutical agent contained within the packaging material. The article of manufacture may also contain two or more sub-therapeutically effective amounts of a pharmaceutical composition, which together act synergistically to result in amelioration of tissue damage due to coronary vascular occlusion.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent.

In preferred embodiments, the packaging material includes a label that is a tangible expression describing the contents of the article of manufacture and the use of the pharmaceutical agent contained therein.

The pharmaceutical agent in an article of manufacture is any of the compositions of the present invention suitable for providing a Src family tyrosine kinase inhibitor, formulated into a pharmaceutically acceptable form as described herein according to the disclosed indications. Suitable Src family tyrosine kinase inhibitors for purposes of the present invention include chemical inhibitors of Src, including the pyrazolopyrimidine class of Src family tyrosine kinase inhibitors, such as 4-amino-5-(4-methylphenyl)-7-(t-butyl)pyrazolo[3,4-d-]pyrimidine, 4-amino-5-(4-chlorophenyl)-7-(t-butyl)pyrazolo[3,4-d-]pyrimidine, and the like; the macrocyclic dienone class of Src family tyrosine kinase inhibitors, such as Radicicol R2146, Geldanamycin, Herbimycin A, and the like; the pyrido[2,3-d]pyrimidine class of Src family tyrosine kinase inhibitors, such as PD173955, and the like; the 4-anilino-3-quinolinecarbonitrile class of Src family tyrosine kinase inhibitors, such as SKI-606, and the like; and mixtures thereof. The article of manufacture contains an amount of pharmaceutical agent sufficient for use in treating a condition indicated herein, either in unit or multiple dosages.

The packaging material comprises a label which indicates the use of the pharmaceutical agent contained therein, e.g., for treating conditions assisted by the inhibition of vascular permeability increase, and the like conditions disclosed herein. The label can further include instructions for use and related information as may be required for marketing. The packaging material can include container(s) for storage of the pharmaceutical agent.

### EXAMPLES

The following examples relating to this invention are illustrative and should not, of course, be construed as specifically limiting the invention. Moreover, such variations of the invention, now known or later developed, which would be within the purview of one skilled in the art are to be considered to fall within the scope of the present invention hereinafter claimed.

### Example 1

### VEGF-Mediated VP Activity Depends on Src and Yes, But not Fyn

The specificity of the Src requirement for VP was explored by examining the VEGF-induced VP activity associated with SFKs such as Fyn or Yes, which, like Src, are known to be expressed in endothelial cells (Bull et al., FEBS Letters, 361:41-44 (1994); Kiefer et al., Curr. Biol. 4:100-109 (1994)). It was confirmed that these three SFKs were expressed equivalently in the aortas of wild-type mice. Like src^{-/-} mice, animals deficient in Yes were also defective in VEGF-induced VP. However, surprisingly, mice lacking Fyn retained a high VP in response to VEGF that was not significantly different from control animals. The disruption of VEGF-induced VP in src^{-/-} or yes^{-/-} mice demonstrates that the kinase activity of specific SFKs is essential for VEGF-mediated signaling event leading to VP activity but not angiogenesis.

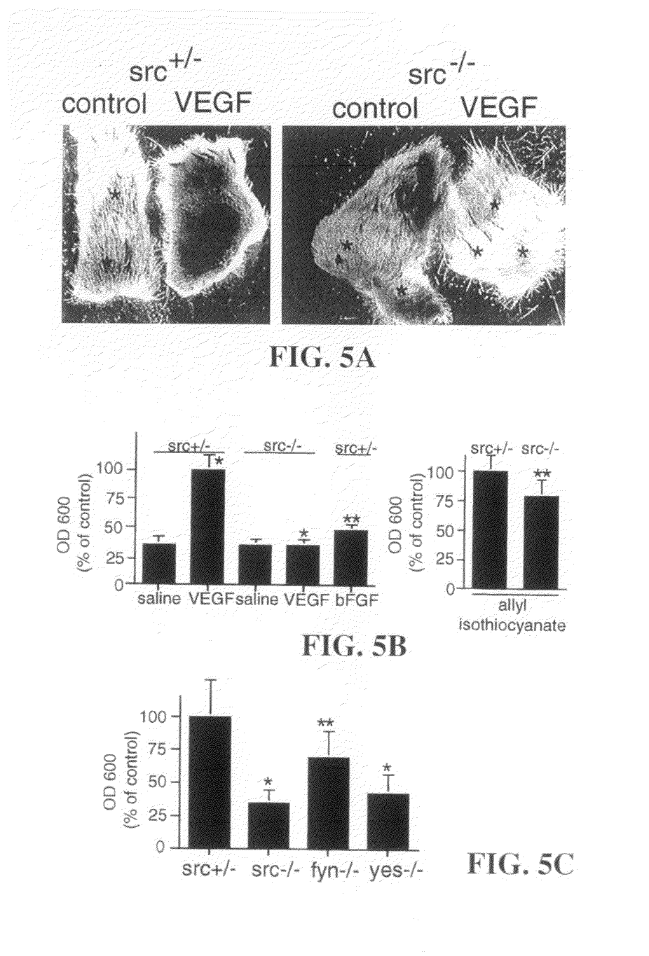

The vascular permeability properties of VEGF in the skin of src^{+/-} (FIG. 5A, left panel) or src^{-/-} (FIG. 5A, right panel) mice was determined by intradermal injection of saline or VEGF (400 ng) into mice that have been intravenously injected with Evan's blue dye. After 15 min, skin patches were photographed (scale bar, 1 mm). The stars indicate the injection sites. The regions surrounding the injection sites of VEGF, bFGF or saline were dissected, and the VP was quantitatively determined by elution of the Evan's blue dye in formamide at 58° C. for 24 hr, and the absorbance measured at 500 nm (FIG. 5B, left graph). The ability of an inflammation mediator (allyl isothiocyanate), known to induce inflammation related VP, was tested in src^{+/-} or src^{-/-} mice (FIG. 5B, right).

The ability of VEGF to induce VP was compared in src^{-/-}, fyn^{-/-}, or yes^{-/-} mice in the Miles assay (FIG. 5C). Data for each of the Miles assays are expressed as the mean ±SD of triplicate animals. src^{-/-} and yes^{-/-} VP defects compared to control animals were statistically significant (*p<0.05, paired t test), whereas the VP defects in neither the VEGF-treated fyn^{-/-} mice nor the allyl isothiocyanate treated src^{+/-} mice were statistically significant (**p<0.05).

### Example 2

### Src Family Tyrosine Kinase Inhibitor Treated Mice, and Src −/− Mice Show Reduced Tissue Damage Associated with Trauma or Injury to Blood Vessels than Untreated Wild-Type Mice

Inhibitors of the Src family kinases reduce pathological vascular leakage and permeability after a vascular injury or disorder such as a stroke. The vascular endothelium is a dynamic cell type that responds to many cues to regulate processes such as the sprouting of new blood vessels during angiogenesis of a tumor, to the regulation of the permeability of the vessel wall during stroke-induced edema and tissue damage.

Reduction of vascular permeability in two mouse stroke models, by drug inhibition of the Src pathway, is sufficient to inhibit brain damage by reducing ischemia-induced vascular leak. Furthermore, in mice genetically deficient in Src, which have reduced vascular leakage/permeability, infarct volume is also reduced. The combination of the synthetic Src inhibitor data, with the supporting genetic evidence of reduced the vascular leakage in stroke and other related models demonstrates the physiological relevance of this approach in reducing brain damage following strokes. Inhibition of these pathways with a range of available Src family kinase inhibitors of these signaling cascades has the therapeutic benefit of mitigating brain damage from vascular permeability-related tissue damage.

Two different methods for induction of focal cerebral ischemia were used. Both animal models of focal cerebral ischemia are well established and widely used in stroke research. Both models have been previously used to investigate the pathophysiology of cerebral ischemia as well as to test novel antistroke drugs.

(a) Mice were anesthetized with 2,2,2,-tribromoethanol (AVERTIN™) and body temperature was maintained by keeping the animal on a heating pad. An incision was made between the right ear and the right eye. The scull was exposed by retraction of the temporal muscle and a small burr hole was drilled in the region over the middle cerebral artery (MCA). The meninges were removed, and the right MCA was occluded by coagulation using a heating filament. The animals were allowed to recover and were returned to their cages. After 24 hours, the brains were perfused, removed and cut into 1 mm cross-sections. The sections were immersed in a 2% solution of 2,3,5-triphenyltetrazolium chloride (TTC), and the infarcted brain area was identified as unstained (white) tissue surrounded by viable (red) tissue. The infarct volume was defined as the sum of the unstained areas of the sections multiplied by their thickness.

Mice deficient in Src (Src−/−) were used to study the role of Src in cerebral ischemia. Src+/− mice served as controls. We found that in Src−/− mice the infarct volume was reduced from 26±10 mm³ to 16±4 mm³ in controls 24 hours after the insult. The effect was even more pronounced when C57B16 wild-type mice were injected with 1.5 mg/kg AGL1872 intraperitoneally (i.p.) 30 min after the vessel occlusion. The infarct size was reduced from 31±12 mm³ in the untreated group to 8±2 mm³ in the AGL1872-treated group.

(b) In a second model of focal cerebral ischemia the MCA was occluded by placement of an embolus at the origin of the MCA. A single intact fibrin-rich 24 hour old homologous clot was placed at the origin of the MCA using a modified PE-50 catheter. Induction of cerebral ischemia was proven by the reduction of cerebral blood flow in the ipsilateral hemisphere compared to the contralateral hemisphere. After 24 hours the brains were removed, serial sections were prepared and stained with hematoxylin-eosin (HE). Infarct volumes were determined by adding the infarct areas in serial HE sections multiplied by the distance between each section.

The dosage of AGL1872 used in this study (1.5 mg/kg i.p.) was empirically chosen. It is known that VEGF is first expressed about 3 hours after cerebral ischemia in the brain with a maximum after 12 to 24 hours. In this study AGL1872 was given 30 min after the onset of the infarct to completely block VEGF-induced vascular permeability increase. According to the time course of typical VEGF expression, a potential therapeutical window for the administration of Src-inhibitors can be up to 12 hours after the stroke. In diseases associated with a sustained increase in vascular permeability a chronic administration of the Src inhibiting drug is appropriate.

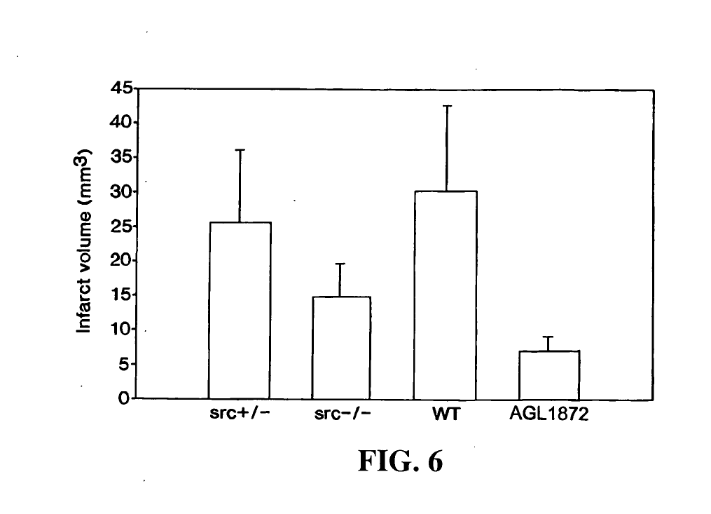

FIG. 6 is a graph which depicts the comparative results of averaged infarct volume (mm³) in mouse brains after injury, where mice were heterogeneous Src (Src +/−), dominant negative Src mutants (Src −/−), wild type mice (WET), or wild type mice treated with 1.5 mg/kg AGL1872.

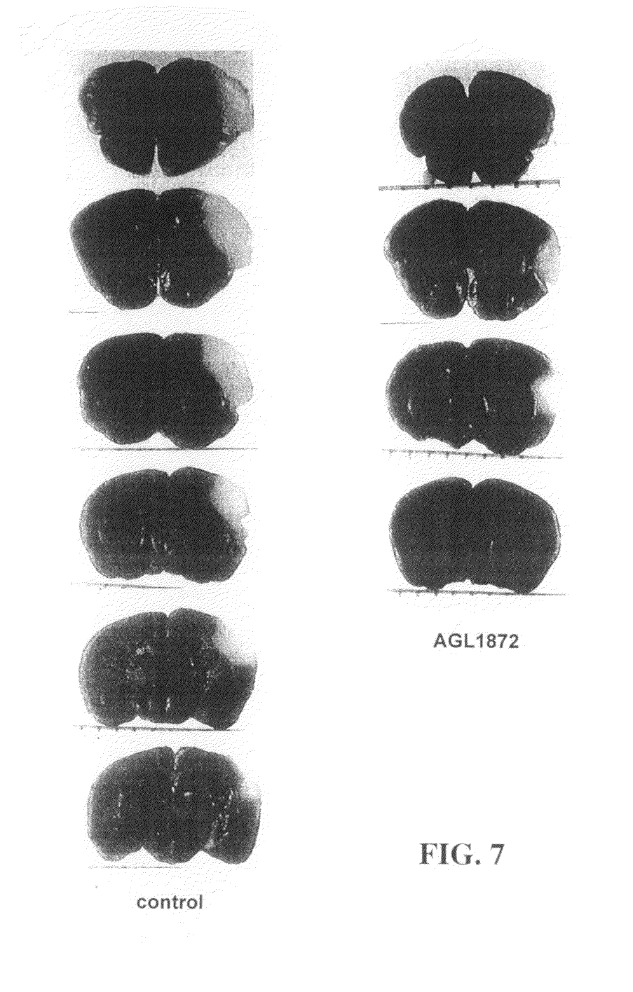

FIG. 7 illustrates sample sequential MRI scans of isolated perfused mouse brain after treatment to induce CNS injury, where the progression of scans in the AGL1872 treated animal (right) clearly shows less cerebral infarct than the progression of scans in the control untreated animal (left).

### Example 3

### Src Family Tyrosine Kinase Inhibitor Treated Rats, and Src −/− Mice Show Reduced Tissue Damage Associated with Trauma or Injury to Coronary Blood Vessels than Untreated Wild-Type Mice

Myocardial ischemia was induced by ligating the left anterior descending coronary artery in Sprague-Dawley rats. The affected heart tissue was contacted with a chemical Src family tyrosine kinase inhibitor by intraperitoneal (i.p.) injections of the pyrazolopyrimidine class Src family tyrosine kinase inhibitor AGL1872 or SKI-606 after the induction of ischemia. High resolution magnetic resonance imaging (MRI), dry weight measurements, infarct size, heart volume, and area at risk were determined 24 hours postoperatively. Survival rates and echocardiography were determined at 4 weeks postoperatively in the rats receiving i.p. injections of the inhibitor at a dosage of about 1.5 mg/kg following myocardial infarction (MI).

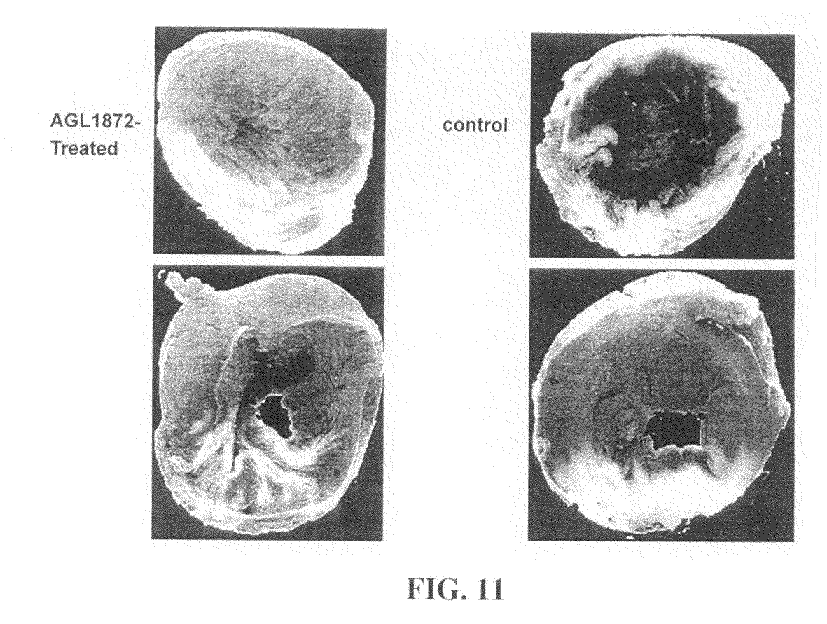

FIG. 11 shows photomicrographic images of treated (left) and control (right) rat heart tissue stained with an eosin dye (vital stain). The control tissue (upper right image) shows a large area of necrosis at the periphery of the tissue. In contrast, the treated tissue (upper left image) shows very little necrotic tissue.

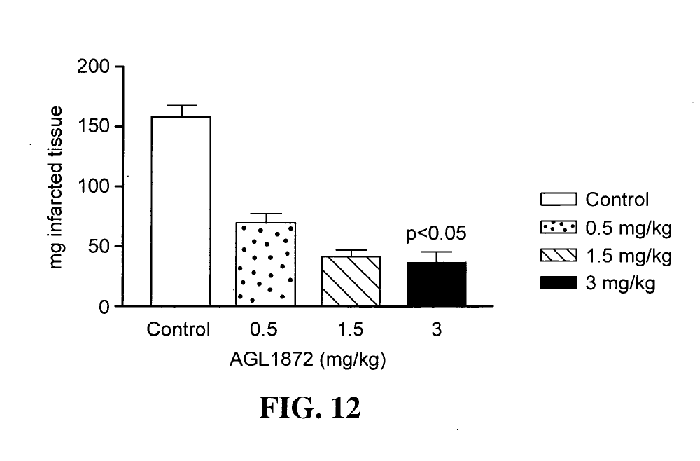

FIG. 12 shows a bar graph of infarct size after 24 hours post treatment (in mg of tissue) as a function of inhibitor (AGL1872) concentration. An optimal level of inhibition was achieved at a dosage of about 1.5 mg/kg. A dosage of about 3 mg/kg did not result in any significant reduction in infarct size.

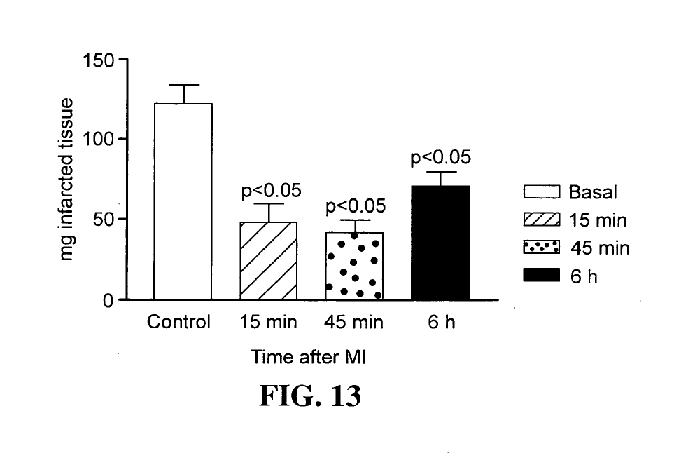

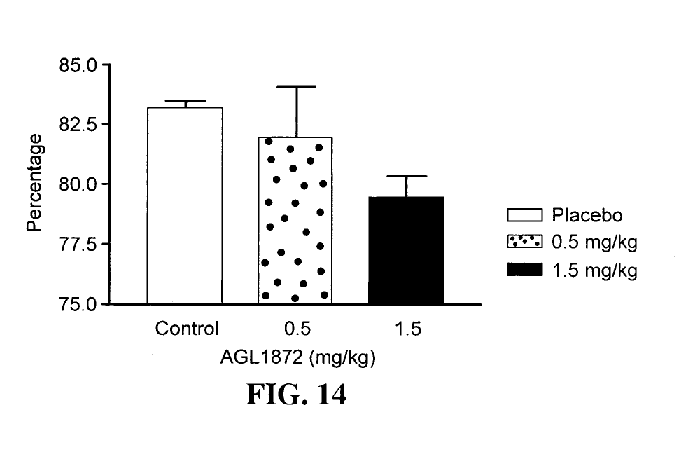

Treatment with the Src family tyrosine kinase inhibitor resulted in a decrease in infarct size and area at risk in a dose dependent manner within 24 hours postoperative. A maximum inhibition of about 68% (p<0.05) in infarct size was achieved at a dosage of about 1.5 mg/kg of the inhibitor delivered about 45 minutes after induction of ischemia (FIG. 13). The inhibitor was also effective when given about 6 hours after induction of ischemia, resulting in a decrease of about 42% in the infarct size (p<0.05). Src inhibition did not interfere with VEGF expression in the ischemic tissues as determined by immunohistochemical analysis. Reduced infarct size was accompanied by decreased myocardial water content (about 5%+/−1.3%; p<0.05) and a reduction in volume of the edematous tissue as detected by MRI, indicating that the beneficial effect of Src inhibition was associated with prevention of VEGF-mediated VP (FIG. 14). Fractional shortening, as assessed by echocardiography at about 4 weeks postoperatively, was about 29% in the control and about 34% in the treated rats (p<0.05). Significantly, the four week survival rate was unexpectedly high (100%) for the treated rats, relative to about 63% for the control rats.

To precisely monitor edema in-vivo, we used high-resolution magnetic resonance imaging (MRI) to evaluate the cardiac tissue of rats that were treated with or without the Src inhibitors AGL1872 or SKI-606 following permanent left anterior descending (LAD) occlusion. Because of their increased water content, edematous regions are expected to have a longer T₂ relaxation than nonedamatous regions. To quantify edema, regions with T₂>49 ms (greater than two standard deviations above the mean of normally perfused myocardium) were delineated. One hour after the onset of ischemia, T₂-weighted signaling indicated Src inhibition did not influence the initial cytotoxic edema. However, after 24 hours, computed T₂ maps revealed a 47% reduction in infarct-related myocardial edema by AGL1872 compared with vehicle (n=2 AGL1872 group, n=1 vehicle group). This result correlates with myocardial water content computed ex-vivo using wet/dry weights of nonischemic myocardium. AGL1872 provided dose-dependent decreases in edema and infarct size, with a maximum decrease at 1.5 mg/kg (n>5 each group, P<0.001). SKI-606 also provided significant reduction of infarct size when administered following permanent occlusion in the mouse and rat. To evaluate the kinetics of this response, AGL1872 was administered at various times following occlusion. While maximum benefit (50% smaller infarct size) was achieved with administration 45 minutes following occlusion, treatment after 6 hours still yielded 25% protection (n=5 each group, P<0.05).

Echocardiography revealed Src inhibition offers significant preservation of fractional shortening and diastolic left ventricular (LV) diameter over 4 weeks compared with untreated rats, indicating that contractile function in the rescued tissue was preserved long term. Src inhibition also provided a favorable effect on systolic LV diameter and regional wall motion (Table 1). Treatment with the SKI-606 Src inhibitor also favorably impacted fractional shortening and regional wall motion score (n=7 each group, P<0.01). To evaluate survival after MI, we used 2-year-old C57 black mice as a model characterized by considerably mortality (>40%) after LAD ligation. Administration of AGL1872 (1.5 mg/kg) 45 minutes post-MI increased survival compared with control within the first 4 weeks (91.7% vs. 58.3%, respectively, n=12 each group), demonstrating a long term therapeutic effect of Src inhibition.

TABLE 1
Functional Recovery Following MI: Echocardiography

| | Control | AGL 1872 | % Improvement | P-Value |
|---|---|---|---|---|
| LV diameter, | 0.93 ± 0.02 | 0.82 ± 0.02 | 11 | 0.01 |
| diastole (mm) | | | | |
| LV diameter, | 0.71 ± 0.03 | 0.59 ± 0.04 | 16 | 0.03 |
| systole (mm) | | | | |
| Fractional | 23.8 ± 1.7 | 32.8 ± 3.2 | 38 | 0.03 |
| shortening (%) | | | | |
| Regional wall | 26.9 ± 0.8 | 24.0 ± 0.5 | 9 | 0.01 |
| motion score | | | | |
| # Rats per | 8 | 8 | | |
| group | | | | |

Chronic myocardial fibrosis occurs following infarction and is a direct reflection of extent of tissue necrosis following MI. To evaluate the effect of Src inhibition on fibrosis 4 weeks post-MI in rats, histopathological analysis of fibrotic tissue was performed using elastic trichrome staining. Src inhibition contributed to a 52% decrease in LV fibrotic tissue compared with control (19.1±2.2% vs. 40.0±3.0%, n=4 each group, P<0.01). Consistently better reservation of myocardial fibers and LV architecture was observed among the samples which received the Src inhibitor, indicating that Src inhibition contributes to a long term protective effect on the myocardium post-MI.

To establish the effectiveness of Src inhibition following transient ischemia, rats were subjected to occlusion followed by reperfusion, and then evaluated for ventricular function and infarct size after 24 hours. Src inhibition by AGL1872 preserved left ventrical (LV) fractional shortening and reducing infarct size compared to controls (n=4 each group, P<0.05). The 18% reduction in infarct size following ischemia-reperfusion compares to a 50% decrease following permanent occlusion in which the hypoxic stimulus driving VEGF expression is maintained. In addition, SKI-606 (5 mg/kg) provided a 43% decrease in infarct size in the ischemia-reperfusion model (n=5 each group, P<0.01). Collectively, this data demonstrates a beneficial effect of Src inhibition following transient ischemia.

### Example 4

### Effect of MI on Vascular Integrity and Myocyte Viability in Peri-Infarct Zone

Since VEGF expression increases primarily in the peri-infarct zone, the ultrastructural effects of Src inhibition on small vessels in this region was investigated 3-24 hours post-MI. Table 2 provides a summary of observations for 250 blood vessels examined per group using transmission electron microscopy. In contrast to normal myocardial tissue numerous examples of damage in the peri-infarct zone were observed in the infarct affected tissue. Extravasated blood cells (RBC, platelets, and neutrophils) were present in the interstitium, apparently having escaped from nearby vessels. Some endothelial cells (EC) were swollen and occluded part of the vessel lumen, often appearing electron-lucent and containing many caveolae. Large round vacuoles were present in the endothelium, often several times larger than the EC thickness. Myocyte injury increased with time following MI and varied between adjacent cells, identifiable as mitochondrial rupture, disordered mitochondrial cristae, intracellular edema, and myofilament disintegration. The most affected myocytes were often adjacent to injured blood vessels or free blood cells. We frequently observed neutrophils 24 hours after MI, which participate in the acute response to injury and may contribute to VEGF production.

TABLE 2
Ultrastructural observations in mouse cardiac tissue following MI or
VEGF injection

| | | Platelet | | |
|---|---|---|---|---|
| | ECBarrier | Activation | EC | Cardiac |
| | Dysfunction | and Adhesion | Injury | Damage |
| 3 hr MI | 18 | 36 | 31 | 22 |
| 3 hr MI + AGL1872 | 2 | 11 | 14 | 2 |
| 24 hr MI | 5 | 7 | 34 | 45 |
| 24 hr MI + AGL1872 | 0 | 1 | 15 | 9 |
| Control | 0 | 0 | 1 | 0 |
| VEGF, pp60Src +/+ | 24 | 18 | 33 | 16 |
| VEGF, pp60Src +/+ | 0 | 0 | 0 | 0 |

For each group, left ventricular tissue was examined for 4 hours (approximately 250 microvessels) on a transmission electron microscope and observations were counted and grouped according to:
(a) EC Barrier Dysfunction: Gaps, Fenestration, Extravasated blood cells;
(b) Platelet Activation/Adhesion: Platelets, Degranulated platelets, Platelet clusters, Platelet adhesion to ECM;
(c) EC Injury: Electron-lucent EC, Swollen EC, Large EC vacuoles, Occluded vessel lumen; and
(d) Cardiac Damage: Mitochondrial swelling, Disordered cristae, Myofilament disintegration.

Three hours following MI, gaps were frequently observed between adjacent EG, which could explain the extravasation of blood cells to the surrounding interstitial space. Surprisingly, many of the gaps were plugged by platelets. Some platelets contacted the basal lamina exposed between EC while in other cases the basal lamina also appeared to be disrupted. Some of the platelets were degranulated and may have potentiated the further activation, adhesion, and aggregation of circulating platelets. While these platelet plugs may have prevented further vascular leak, they could inadvertently have contributed to decreased perfusion in small vessels via microthrombi formation, which could lead to further ischemia-related tissue disease.

### Example 5

### MI and Systematic VEGF Injection Produce a Similar Vascular Response

To determine the contribution of VEGF to the complex pathology or MI, VEGF was intravenously injected into normal mice and cardiac tissue was evaluated at the ultrastructural level after 30 minutes. Surprisingly, the extent of VEGF-induced endothelial barrier dysfunction and vessel injury was comparable to that seen in the peri-infarct zone post-MI (Table 2). Considerable platelet adhesion was observed to the EC basement membrane as well as myocyte damage. Similar evidence of damage in the brain was found following systemic VEGF injection suggesting these effects may be systemic. These results indicate that VEGF-mediated VP parallels many of the vascular effects following MI.

To determine whether VEGF is sufficient to mediate longer term pathology associated with MI, mice were injected four times with VEGF over the course of 2 hours. This treatment created damage similar to that observed 24 hours post-MI. Platelet adhesion, neutrophils, and significant myocyte damage were found, as well as numerous electron-lucent EC, many of which were swollen to occlude the vessel lumen. Taken together, 30 minutes exposure to VEGF were sufficient to induce an ultrastructure similar to that observed after 3 hours of MI, by which time VEGF expression significantly increased in the peri-infarct zone. Longer term VEGF exposure elicited vascular remodeling similar to that seen in tissues 24 hours after MI.

The fact that Src-deficient mice were protected following MI and lacked VP in the skin and brain following local VEGF injection suggests that the Src deficient mice were spared from VEGF-induced VP in the heart. Consistent with the Src inhibitor results, no signs of a vascular response following VEGF injection were seen in the pp60Src−/− mice (Table 2), compared with gaps, platelet activity, affected EC, and extravasated blood cells in wild type mice. The complete blockade of any response suggests that VEGF-mediated Src activity initiates a cascade leading to VP-induced injury during ischemic disease.

### DISCUSSION

In mice, systemic administration of a VE-cadherin antibody caused VP in the heart and lungs, interstitial edema, and focal spots of exposed basement membrane that appear similar at the ultrastructural level with damage observed following VEGF administration. In mouse embryos, β-catenin-null blood vessels contain flattened, fenestrated endothelial cells associated with frequent hemorrhage. Previous in vitro studies have implicated VEGF in the regulation of VE-cadherin function. In EC under flow conditions, VE-cadherin complexes with Flk. To evaluate the VE-cadherin-VEGF complex in vivo, heart lysates were prepared from mice injected with or without VEGF. These lysates were subjected to immunoprecipitation with anti-Flk followed by immunoblotting for VE-cadherin and β-catenin. In control mice, a pre-existing complex between Flk, β-catenin, and VE-cadherin in blood vessels was observed. This complex was rapidly disrupted within 2-5 minutes following VEGF stimulation, and had reassembled by 15 minutes in blood vessels in vivo. The timescale for dissociation of the complex completely paralleled that of Flk, β-catenin, and VE-cadherin phosphorylation and the dissociation of β-catenin from VE-cadherin. These VEGF-mediated events were Src-dependent, since the Flk-cadherin-catenin signaling complex remained intact and phosphorylation of β-catenin and VE-cadherin did not occur in VEGF-stimulated mice pretreated with Src inhibitors. These events were not observed following injection of basic fibroblast growth factor (bFGF), a similar antiogenic growth factor which does not promote vascular permeability.

While a single VEGF injection produced a reversible, rapid, and transient signaling response which returned to baseline by 15 minutes, four VEGF injections (every thirty minutes) produced a prolonged signaling response. For example, dissociation of Flk-catenin and Erk phosphorylation persisted following prolonged VEGF exposure. This model may be applicable to the physiological situation following MI, wherein VEGF expression increases due to hypoxia and persists for days.

Src plays a physiological and molecular role in VP following acute MI or systemic VEGF administration. Poor outcome following MI apparently is due in part to hyperpermeability of the perfused cardiac microvessels surrounding the infarct zone. These vessels are adversely affected by VEGF and undergo a Src-dependent increase in VP which leads to vessel occlusion or collapse, and ultimately to damage of the surrounding myocytes. This is consistent with the persistence of poor tissue perfusion and high mortality that has been documented following MI despite vessel opening during reperfusion. Src inhibition as late as 6 hours post-MI still provides significant protection against VEGF-induced VP, indicating relevance of this approach in a clinical setting. Administration of Src inhibitors following MI appears to limit VP by preventing dissociation of Flk-cadherin-catenin complexes which maintain endothelial barrier function.

Ultrastructural data suggest that the initial effects of VEGF following MI involve opening of endothelial junctions exposing the endothelial basement membrane. Platelets, many of which were degranulated and activated, adhered to these sites. This is of interest since platelets contain VEGF, which when released locally upon platelet activation may augment the VP response. In fact, it is possible that some of the beneficial effects of Src inhibition are due to its effect on platelet activation. It is apparent from the present data that the early events following MI initiate a cascade that results in accumulation of edema, tissue damage which is then followed by fibrosis and remodeling of the heart tissue. It is important to point out that the fibrotic remodeled cardiac tissue is functionally inferior to the normal cardiac tissue. Thus, by limiting the impact of the injury early on, long term benefits due to the need to remodel less of the cardiac tissue can be expected. Since blockade of a single coronary vessel promotes an acute injury that leads to growth of the infarct zone, fibrosis and in some cases death, an early effective intervention in this process may well provide long term protection and benefit.

The present data reveal that a Src inhibitor may well play such a role. Src inhibition maintains the Flk-cadherin-catenin complex and renders endothelial junctions refractory to the permeability-promoting effects of VEGF.

Surprisingly, systemic injection of VEGF produced many of the ultrastructural effects to cardiac blood vessels seen following MI. VEGF alone was sufficient to induce endothelial barrier dysfunction and blood vessel damage in vivo. Likewise, the methods of the present invention, involving blockade of Src with a Src family tyrosine kinase inhibitor not only suppressed these events following MI, but did so after systemic VEGF injection. Src inhibition stabilizes the Flk-cadherin-catenin complex despite VEGF stimulation. Other contributors to VEGF-induced VP may include caveolae or visiculo-vacuolar organelles (VVOs) and fenestrations. These modes of permeability could also be Src-dependent, since pp60Src−/− mice exhibit no signs of permeability following VEGF injection. Alternatively, endothelial gaps, extravasated blood cells, and exposed basement membrane may induce fenestrations and VVOs.

VEGF is expressed in vivo in response to a variety of factors (cytokines, oncogenes, hypoxia) and acts to induce permeability and angiogenesis, as well as endothelial cell proliferation, migration, and protection from apoptosis. Tumors produce large amounts of VEGF which can be detected in the blood stream. In fact, blood vessels within or near tumors share many of the features seen in the present studies following VEGF injection, such as fenestrated endothelium, open interendothelial junctions, and clustered fused caveolae. Serum levels of VEGF in patients with various cancers can range from 100-3000 pg/ml, while local cell or tissue VEGF levels can be 10-100 times higher. In patients following MI, serum VEGF levels have been reported between 100-400 pg/ml, and are higher in patients with acute MI versus stable angina. As for some primary and metastatic tumors, local VEGF levels in the peri-infarct region may well exceed serum levels. The present data may explain findings that some cancer patients have increased thrombotic disease, since increased VEGF accumulation in the circulation would instigate a VP response which attracts platelets and leads to loss of blood flow. In addition, the recently reported observation may account for the pleural effusion and general edema associated with late stage cancer. Thus, blocking Src may have a profound effect on cancer-related edematous disease.

AGL1872, while inhibiting Src family tyrosine kinases, also disrupts a range of other kinases, whereas SKI-606 is reportedly more selective for Src and Yes. Both of these inhibitors showed a similar pattern of biological activity, which mirrored the effects seen in Src-deficient mice. The fact that pharmacological Src inhibitors administered to wild type animals produced the same impact on tissue injury, biochemistry and ultrastructure of the cardiac vessels as that seen in the knockout mice suggests that the effect is primarily due to the EC mediated leakage and is not associated with a genetic predisposition in these animals. Src and Yes, but not Fyn, are essential to the VEGF-mediated VP response and the growth of infarcted tissue following ischemic injury in the brain. Taken together, this data suggests that the beneficial effects of administration of a Src family tyrosine kinase inhibitor following MI are indeed a function of Src kinase inhibition, and implicate pp60Src and pp62Yes as the Src kinases most likely involved.

Essentially identical ultrastructural changes were observed following MI or direct VEGF injection. The fact that VEGF acts primarily on the endothelium and not other cell types suggests that blocking Src within the ECs accounts for the ultrastructural observations. Moreover, most of the changes observed were directly associated with changes in EC cell-cell contact and blood vessel integrity, none of few of which were seen in either Src knockout animals or wild type animals treated with Src inhibitors. Importantly, the role of Src in VP can be attributed to its ability to phosphorylate VE-cadherin and β-catenin, and promote the dissociation of a complex between these junctional proteins with the VEGF receptor, Flk.

The methods of the present invention are well suited for the specific amelioration of VP induced tissue damage, particularly that resulting from myocardial infarction, because the targeted inhibition of Src family tyrosine kinase action focuses inhibition on VP without a long term effect on other VEGF-induced responses which can be beneficial to recovery from injury.

Src appears to regulate tissue damage by influencing VEGF-mediated vasopermeability and thus represents a novel therapeutic target in the pathophysiology of myocardial ischemia. The extent of myocardial damage following coronary artery occlusion can be significantly reduced by acute pharmacological inhibition of Src family tyrosine kinases.

The use of synthetic, relatively small-molecule chemical inhibitors is in general safer and more manageable that the use of the relatively larger proteins. Thus, the former are preferred as therapeutically active agents.

The foregoing specification enables one skilled in the art to practice the invention. Indeed, various modifications of the invention in addition to those shown and described herein will become apparent to those skilled in the art from the foregoing description and fall within the scope of the appended claims.

We claim:

1. A method for treating a mammal suffering from a myocardial infarction comprising administering to the mammal a therapeutically effective amount of a pharmaceutical composition comprising a chemical Src family tyrosine kinase inhibitor.

2. The method of claim 1 wherein the mammal is a human.

3. The method of claim 1 wherein the mammal is a non-human mammal.

4. The method of claim 1 wherein the Src family tyrosine kinase inhibitor is a an inhibitor of Src protein.

5. The method of claim 4 wherein the chemical inhibitor is selected from the group consisting of a pyrazolopyrimidine class Src family tyrosine kinase inhibitor, a macrocyclic dienone class Src family tyrosine kinase inhibitor, a pyrido[2,3-d]pyrimidine class Src family tyrosine kinase inhibitor, a 4-anilino-3-quinolinecarbonitrile class Src family tyrosine kinase inhibitor, and a mixture thereof.

6. The method of claim 5 wherein the pyrazolopyrimidine class Src family tyrosine kinase inhibitor is a member of the group consisting of 4-amino-5-(4-methylphenyl)-7-(t-butyl)pyrazolo[3,4-d-]pyrimidine, 4-amino-5-(4-chlorophenyl)-7-(t-butyl)pyrazolo[3,4-d-]pyrimidine, and a mixture thereof.

7. The method of claim 5 wherein the macrocyclic dienone class Src family tyrosine kinase inhibitor is a member of the group consisting of Geldanamycin, Herbimycin A, Radicicol R2146, and a mixture thereof.

8. The method of claim 5 wherein the pyrido[2,3-d]pyrimidine class Src family tyrosine kinase inhibitor is PD173955.

9. The method of claim 5 wherein the 4-anilino-3-quinolinecarbonitrile class Src family tyrosine kinase inhibitor is SKI-606.

10. The method of claim 1 wherein the pharmaceutical composition is administered to the mammal by intraperitoneal injection.

11. The method of claim 1 wherein the pharmaceutical composition is administered to the mammal by intravenous injection.

12. The method of claim 1 wherein the pharmaceutical composition is administered to the mammal within about 6 hours after the myocardial infarction.

13. The method of claim 1 wherein the pharmaceutical composition is administered to the mammal within about 24 hours after the myocardial infarction.

14. A method for prophylactic treatment of a mammal at risk of myocardial infarction, the method comprising administering to the mammal a prophylactic amount of a pharmaceutical composition comprising a chemical Src family tyrosine kinase inhibitor.

15. The method of claim 14 wherein the mammal is a non-human mammal.

16. The method of claim 14 wherein the mammal is a human.

17. The method of claim 14 wherein the pharmaceutical composition is orally administered to the mammal.

18. The method of claim 14 wherein the pharmaceutical composition is parenterally administered to the mammal.

19. The method of claim 15 wherein the Src family tyrosine kinase inhibitor is a pyrazolopyrimidine class Src family tyrosine kinase inhibitor.

20. The method of claim 19 wherein the pyrazolopyrimidine class Src family tyrosine kinase inhibitor is selected from the group consisting of 4-amino-5-(4-methylphenyl)-7-(t-butyl)pyrazolo[3,4-d-]pyrimidine, 4-amino-5-(4-chlorophenyl)-7-(t-butyl)pyrazolo[3,4-d-]pyrimidine, and a mixture thereof.

21. The method of claim 14 wherein the Src family tyrosine kinase inhibitor is a 4-anilino-3-quinolinecarbonitrile compound.

22. The method of claim 5 wherein the pyrazolopyrimidine class Src family tyrosine kinase inhibitor is 4-amino-5-(4-methylphenyl)-7-(t-butyl)-pyrazolo[3,4-d-]pyrimidine.

23. The method of claim 19 wherein the pyrazolopyrimidine class Src family tyrosine kinase inhibitor is 4-amino-5-(4-methylphenyl)-7-(t-butyl)-pyrazolo[3,4-d-]pyrimidine.


SEQUENCE LISTING (4 sequences)

SEQ ID NO: 1 - DNA, 2187 bp, homo sapiens
  CDS (134)...(1486)
gcgccgcgtcccgcaggccgtgatgccgcccgcgcggaggtggcccggaccgcagtgccccaagagagctctaatggtaccaagtgacaggttggctttactgtgactcggggacgccagagctcctgagaagatgtcagcaatacaggccgcctggccatccggtacagaatgtattgccaagtacaacttccacggcactgccgagcaggacctgcccttctgcaaaggagacgtgctcaccattgtggccgtcaccaaggaccccaactggtacaaagccaaaaacaaggtgggccgtgagggcatcatcccagccaactacgtccagaagcgggagggcgtgaaggcgggtaccaaactcagcctcatgccttggttccacggcaagatcacacgggagcaggctgagcggcttctgtacccgccggagacaggcctgttcctggtgcgggagagcaccaactaccccggagactacacgctgtgcgtgagctgcgacggcaaggtggagcactaccgcatcatgtaccatgccagcaagctcagcatcgacgaggaggtgtactttgagaacctcatgcagctggtggagcactacacctcagacgcagatggactctgtacgcgcctcattaaaccaaaggtcatggagggcacagtggcggcccaggatgagttctaccgcagcggctgggccctgaacatgaaggagctgaagctgctgcagaccatcgggaagggggagttcggagacgtgatgctgggcgattaccgagggaacaaagtcgccgtcaagtgcattaagaacgacgccactgcccaggccttcctggctgaagcctcagtcatgacgcaactgcggcatagcaacctggtgcagctcctgggcgtgatcgtggaggagaagggcgggctctacatcgtcactgagtacatggccaaggggagccttgtggactacctgcggtctaggggtcggtcagtgctgggcggagactgtctcctcaagttctcgctagatgtctgcgaggccatggaatacctggagggcaacaatttcgtgcatcgagacctggctgcccgcaatgtgctggtgtctgaggacaacgtggccaaggtcagcgactttggtctcaccaaggaggcgtccagcacccaggacacgggcaagctgccagtcaagtggacagcccctgaggccctgagagagaagaaattctccactaagtctgacgtgtggagtttcggaatccttctctgggaaatctactcctttgggcgagtgccttatccaagaattcccctgaaggacgtcgtccctcgggtggagaagggctacaagatggatgcccccgacggctgcccgcccgcagtctatgaagtcatgaagaactgctggcacctggacgccgccatgcggccctccttcctacagctccgagagcagcttgagcacatcaaaacccacgagctgcacctgtgacggctggcctccgcctgggtcatgggcctgtggggactgaacctggaagatcatggacctggtgcccctgctcactgggcccgagcctgaactgagccccagcgggctggcgggcctttttcctgcgtcccagcctgcacccctccggccccgtctctcttggacccacctgtggggcctggggagcccactgaggggccagggaggaaggaggccacggagcgggaggcagcgccccaccacgtcgggcttccctggcctcccgccactcgccttcttagagttttattcctttccttttttgagattttttttccgtgtgtttattttttattatttttcaagataaggagaaagaaagtacccagcaaatgggcattttacaagaagtacgaatcttatttttcctgtcctgcccgtgagggtgggggggaccgggcccctctctagggacccctcgccccagcctcattccccattctgtgtcccatgtcccgtgtctcctcggtcgccccgtgtttgcgcttgaccatgttgcactgtttgcatgcgcccgaggcagacgtctgtcaggggcttggatttcgtgtgccgctgccacccgcccacccgccttgtgagatggaattgtaataaaccacgccatgaggacaccgccgcccgcctcggcgcttcctccaccgaaaaaaaaaaaaaaaaaa

SEQ ID NO: 2 - protein, 450 aa, homo sapiens
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 3 - DNA, 4517 bp, homo sapiens
  CDS (208)...(1839)
gcggagccaaggcacacgggtctgacccttgggccggcccggagcaagtgacacggaccggtcgcctatcctgaccacagcaaagcggcccggagcccgcggaggggacctgacgggggcgtaggcgccggaaggctgggggccccggagccgggccggcgtggcccgagttccggtgagcggacggcggcgcgcgcagatttgataatgggctgcattaaaagtaaagaaaacaaaagtccagccattaaatacagacctgaaaatactccagagcctgtcagtacaagtgtgagccattatggagcagaacccactacagtgtcaccatgtccgtcatcttcagcaaagggaacagcagttaatttcagcagtctttccatgacaccatttggaggatcctcaggggtaacgccttttggaggtgcatcttcctcattttcagtggtgccaagttcatatcctgctggtttaacaggtggtgttactatatttgtggccttatatgattatgaagctagaactacagaagacctttcatttaagaagggtgaaagatttcaaataattaacaatacggaaggagattggtgggaagcaagatcaatcgctacaggaaagaatggttatatcccgagcaattatgtagcgcctgcagattccattcaggcagaagaatggtattttggcaaaatggggagaaaagatgctgaaagattacttttgaatcctggaaatcaacgaggtattttcttagtaagagagagtgaaacaactaaaggtgcttattccctttctattcgtgattgggatgagataaggggtgacaatgtgaaacactacaaaattaggaaacttgacaatggtggatactatatcacaaccagagcacaatttgatactctgcagaaattggtgaaacactacacagaacatgctgatggtttatgccacaagttgacaactgtgtgtccaactgtgaaacctcagactcaaggtctagcaaaagatgcttgggaaatccctcgagaatctttgcgactagaggttaaactaggacaaggatgtttcggcgaagtgtggatgggaacatggaatggaaccacgaaagtagcaatcaaaacactaaaaccaggtacaatgatgccagaagctttccttcaagaagctcagataatgaaaaaattaagacatgataaacttgttccactatatgctgttgtttctgaagaaccaatttacattgtcactgaatttatgtcaaaaggaagcttattagatttccttaaggaaggagatggaaagtatttgaagcttccacagctggttgatatggctgctcagattgctgatggtatggcatatattgaaagaatgaactatattcaccgagatcttcgggctgctaatattcttgtaggagaaaatcttgtgtgcaaaatagcagactttggtttagcaaggttaattgaagacaatgaatacacagcaagacaaggtgcaaaatttccaatcaaatggacagctcctgaagctgcactgtatggtcggtttacaataaagtctgatgtctggtcatttggaattctgcaaacagaactagtaacaaagggccgagtgccatatccaggtatggtgaaccgtgaagtactagaacaagtggagcgaggatacaggatgccgtgccctcagggctgtccagaatccctccatgaattgatgaatctgtgttggaagaaggaccctgatgaaagaccaacatttgaatatattcagtccttcttggaagactacttcactgctacagagccacagtaccagccaggagaaaatttataattcaagtagcctattttatatgcacaaatctgccaaaatataaagaacttgtgtagattttctacaggaatcaaaagaagaaaatcttctttactctgcatgtttttaatggtaaactggaatcccagatatggttgcacaaaaccacttttttttccccaagtattaaactctaatgtaccaatgatgaatttatcagcgtatttcagggtccaaacaaaatagagctaagatactgatgacagtgtgggtgacagcatggtaatgaaggacagtgaggctcctgcttatttataaatcatttcctttctttttttccccaaagtcagaattgctcaaagaaaattatttattgttacagataaaacttgagagataaaaagctataccataataaaatctaaaattaaggaatatcatgggaccaaataattccattccagttttttaaagtttcttgcatttattattctcaaaagttttttctaagttaaacagtcagtatgcaatcttaatatatgctttcttttgcatggacatgggccaggtttttcaaaaggaatataaacaggatctcaaacttgattaaatgttagaccacagaagtggaatttgaaagtataatgcagtacattaatattcatgttcatggaactgaaagaataagaactttttcacttcagtccttttctgaagagtttgacttagaataatgaaggtaactagaaagtgagttaatcttgtatgaggttgcattgattttttaaggcaatatataattgaaactactgtccaatcaaaggggaaatgttttgatctttagatagcatgcaaagtaagacccagcattttaaaagcccttttttaaaaactagacttcgtactgtgagtattgcttatatgtccttatggggatgggtgccacaaatagaaaatatgaccagatcagggacttgaatgcacttttgctcatggtgaatatagatgaacagagaggaaaatgtatttaaaagaaatacgagaaaagaaaatgtgaaagttttacaagttagagggatggaaggtaatgtttaatgttgatgtcatggagtgacagaatggctttgctggcactcagagctcctcacttagctatattctgagactttgaagagttataaagtataactataaaactaatttttcttacacactaaatgggtatttgttcaaaataatgaagttatggcttcacattcattgcagtgggatatggtttttatgtaaaacatttttagaactccagttttcaaatcatgtttgaatctacattcacttttttttgttttcttttttgagacggagtctcgctctgccgcccaggctggagtgcagtggcgcgatctcggctcactgcaagctctgcctcccaggttcacaccattctcctgcctcagcctcccgagtagctgggactacaggtgcccaccaccacgcctggctagttttttgtatttttagtagagacgcagtttcaccgtgttagccaggatggtctcgatctcctgaccttgtgatctgcccgcctcggcctcccaaagtgctgggattacaggtgtgagccaccgcgcccagcctacattcacttctaaagtctatgtaatggtggtcattttttcccttttagaatacattaaatggttgatttggggaggaaaacttattctgaatattaacggtggtgaaaaggggacagtttttaccctaaagtgcaaaagtgaaacatacaaaataagactaatttttaagagtaactcagtaatttcaaaatacagatttgaatagcagcattagtggtttgagtgtctagcaaaggaaaaattgatgaataaaatgaaggtctggtgtatatgttttaaaatactctcatatagtcacactttaaattaagccttatattaggcccctctattttcaggatataattcttaactatcattatttacctgattttaatcatcagattcgaaattctgtgccatggcgtatatgttcaaattcaaaccatttttaaaatgtgaagatggacttcatgcaagttggcagtggttctggtactaaaaattgtggttgttttttctgtttacgtaacctgcttagtattgacactctctaccaagagggtcttcctaagaagagtgctgtcattatttcctcttatcaacaacttgtgacatgagattttttaagggctttatgtgaactatgatattgtaatttttctaagcatattcaaaagggtgacaaaattacgtttatgtactaaatctaatcaggaaagtaaggcaggaaaagttgatggtattcattaggttttaactgaatggagcagttccttatataataacaattgtatagtagggataaaacactaacaatgtgtattcattttaaattgttctgtatttttaaattgccaagaaaaacaactttgtaaatttggagatattttccaacagcttttcgtcttcagtgtcttaatgtggaagttaacccttaccaaaaaaggaagttggcaaaaacagccttctagcacacttttttaaatgaataatggtagcctaaacttaatatttttataaagtattgtaatattgttttgtggataattgaaataaaaagttctcattgaatgcacc

SEQ ID NO: 4 - protein, 543 aa, homo sapiens
MGCIKSKENKSPAIKYRPENTPEPVSTSVSHYGAEPTTVSPCPSSSAKGTAVNFSSLSMTPFGGSSGVTPFGGASSSFSVVPSSYPAGLTGGVTIFVALYDYEARTTEDLSFKKGERFQIINNTEGDWWEARSIATGKNGYIPSNYVAPADSIQAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVCPTVKPQTQGLAKDAWEIPRESLRLEVKLGQGCFGEVWMGTWNGTTKVAIKTLKPGTMMPEAFLQEAQIMKKLRHDKLVPLYAVVSEEPIYIVTEFMSKGSLLDFLKEGDGKYLKLPQLVDMAAQIADGMAYIERMNYIHRDLRAANILVGENLVCKIADFGLARLIEDNEYTARQGAKFPIKWTAPEAALYGRFTIKSDVWSFGILQTELVTKGRVPYPGMVNREVLEQVERGYRMPCPQGCPESLHELMNLCWKKDPDERPTFEYIQSFLEDYFTATEPQYQPGENL